(19) United States
(12) Patent Application Publication
(10) Pub. No.: US 2005/0008660 A1
(43) Pub. Date: Jan. 13, 2005

(54) Compositions and methods for use of alginate dural sealants

(75) Inventors: Daryl R. Kipke, Dexter, MI (US); Timothy A. Becker, Ann Arbor, MI (US); Justin C. Williams, Fitchburg, WI (US); Rio J. Vetter, Ann Arbor, MI (US)

Correspondence Address: RADER, FISHMAN & GRAUER PLLC, 39533 WOODWARD AVENUE, SUITE 140, BLOOMFIELD HILLS, MI, 48304-0610, US

(73) Assignee: The Regents of the University of Michigan

(21) Appl. No.: 10/797,304

(22) Filed: Mar. 10, 2004

Related U.S. Application Data
(60) Provisional application No. 60/453,305, filed on Mar. 10, 2003.

(51) Int. Cl. A61K 31/715

(52) U.S. Cl. 424/400; 514/54

(57) ABSTRACT

The present invention comprises compositions and methods for use of an alginate dural sealant in conjunction with mammalian neurosurgical procedures, including but not limited to, the implantation of neuroprosthetic devices.

### CROSS-REFERENCE TO RELATED APPLICATIONS

This application claims priority based on U.S. Provisional Patent Application No. 60/453,305, filed Mar. 10, 2003, which is hereby incorporated by reference in full.

### FIELD OF THE INVENTION

This invention relates to compositions and methods of use of an improved sealant in conjunction with neurosurgical procedures.

### BACKGROUND OF THE INVENTION

Neuroprosthetic devices or probes are often used to treat or research pathologies of the brain or nervous system. In the development of an optimized neuroprosthetic device, surgical technique is a significant contributor to the success rate of cortically implantable microelectrode arrays. Many developments have been made over the past two decades to optimize the procedures and extend the lifetimes of electrode implants.

Surgical techniques are a critical contributor to the level of success achieved with chronically implanted cortical neuroprosthetic devices and in sealing the dura after any general neurosurgery. Despite these advances, issues such as the handling of the exposed brain and dura, isolating the electrode from movements, and minimizing pathways for infection remain of great concern when completing an electrode implant procedure. Many different factors can contribute to the amount of irritation of the brain surface from a dura incision and/or an implanted device. Such factors include mechanical irritations, infectious pathogens, and dural regrowth, among others.

Currently, some clinicians and researchers may use materials such as compositions prepared from purified pork skin gelatin/water mixtures (e.g., Gelfoam® (Pharmacia & Upjohn Co.)) to close up the surgical site after a craniotomy. Such materials may be water insoluble, off-white, porous, and nonelastic, and their application may be very time-consuming. In addition, several other problems may persist. As some examples, the porous nature of the material may not produce a tight seal with the surrounding bone, allowing a gateway for toxins and infectious pathogens to the brain. Moreover, the absorbent nature of the material may provide a matrix for dural regrowth, which, if of an extended nature, may be a prime failure mode of chronically implanted electrodes, causing the electrodes to pull out of the brain. The material may also not provide sufficient mechanical strength. In addition, when using a flexible electrode, the brain tends to swell out of the craniotomy, due to intracranial pressures. If there is nothing to retard or stop this swelling, extreme edema occurs, resulting in a large herniation of the neural tissue. The material may also be extremely compressible, allowing the brain to swell through the craniotomy until stopped by some rigid material, such as the acrylic head-cap.

Thus there remains a substantial need for sealant materials that address one or more of these problems.

### SUMMARY OF THE INVENTION

The present invention comprises compositions and methods for use of an alginate dural sealant in conjunction with mammalian neurosurgical procedures. In accordance with embodiments of the invention, we have shown that calcium alginate is a biocompatible and mechanically stable material when used as a dural sealant. The invention addresses the unmet needs of the existing art by enhancing and providing ease of application, biocompatibility, high mechanical stability, and transparent clarity of a dural sealant. The invention also inhibits dural regrowth and tissue encapsulation of implanted devices such as neural probes, resulting in sustained long-term neural recordings. Accordingly, the present invention comprises a novel approach to electrode implant stabilization using calcium alginate, which has many inherent properties that are beneficial, including but not limited to, protecting and visualizing implants.

Other aspects and embodiments of the invention will be apparent to those skilled in the art after reviewing the drawings and the detailed description below.

### BRIEF DESCRIPTION OF THE DRAWINGS

FIG. 1A is a representation of alginate structure and reaction mechanism.

FIG. 1B is a representation of general chemical reactions in the presence of divalent calcium ions, the calcium is ionically substituted at the carboxylic site.

FIG. 2 is an illustration of a typical surgical preparation.

FIGS. 3A-C are histology images of rat cortex protected by calcium alginate.

FIG. 4 shows an example of normal EEG when using calcium alginate as a dural sealant.

FIG. 5 is an example of a seizure control using tAMCA, a chemical known to cause a seizure response

FIG. 6 is a photograph taken through a surgical microscope immediately after the administering of a thin layer of calcium alginate to an implanted chronic Michigan electrode.

FIG. 7 are images of a craniomotomy “window” consisting of an ALGEL interface and a glass coverslip, showing transparency clarity up through 28 days.

### DETAILED DESCRIPTION

In some embodiments, without limitation, the present invention comprises an improved dural sealant material in the form of a calcium alginate hydrogel polymer.

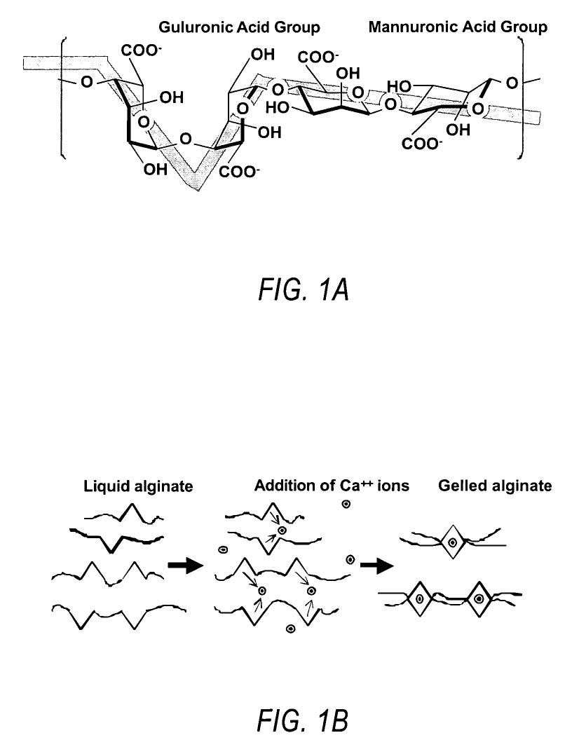

Calcium alginate is a natural sugar-based polymer extracted from seaweed. The polymer is soluble in water, thereby eliminating the need for cytotoxic organic solvents. Calcium alginate is a co-polymer consisting of blocks of mannuronic (M) and guluronic (G) acids in various arrangements (FIG. 1A), resulting in multiple molecular weights. Alginate is a polysaccharide copolymer made of guluronic (G) and mannuronic (M) acid groups. The stereochemistry of the G acid provides reactive carboxylic acid sites. The M acids are not reactive. The guluronic acid sites are active and can react with monovalent and divalent ions, such as sodium and calcium respectively. When reacted with sodium, the ion attaches to the guluronic acid block to form a stable and non-reactive alginate. But in the presence of divalent calcium ions, the higher-free energy calcium is ionically substituted for sodium at the carboxylic site. A second alginate strand can also connect at the divalent calcium ion, forming a link in which the Ca ion attaches two alginate strands together (FIG. 1B). The result is a chain of calcium linked alginate strands that form a solid gel matrix. The resulting alginate gel has non-adhesive, tissue-like mechanical properties and is over 95% water by volume.

The bi-product of the alginate-calcium chloride ionic reaction is saline (sodium and chlorine ions), which is readily accepted by the human body. The concentration of G and M acids (the G/M ratio) contributes to varied structural and biocompatibility characteristics. Alginate's inert tissue-like properties maximize the effective therapy and minimize the potential for adhesion and tissue toxicity.

Calcium alginate's mechanical stability can be increased with increased liquid calcium alginate concentration dissolved in water. However, alginate liquid viscosity increases exponentially with calcium alginate molecular weight and concentration, thereby limiting the concentration of liquid calcium alginate that can be applied with small-bore needles or similar means known to those of ordinary skill. Therefore, in some embodiments, without limitation, liquid calcium alginate is made with a viscosity range of 100-150 cP, to maximize gel stability and minimize viscosity for injection. Calcium alginate concentration can vary from 1 wt % to 2.5 wt % depending on the molecular weight of calcium alginate used. Calcium alginates of similar viscosity but varied concentrations still have similar stability. Calcium alginate has a compressive and fatigue strength range of 20 to 60 kPa. In some embodiments, without limitation, calcium chloride with a concentration of 10 wt % (0.68M) is used to solidify calcium alginate. Calcium chloride solutions ranging from 1 wt % to 30 wt % can also be used. In some embodiments, the invention comprises the application of a calcium alginate composition by filling the craniotomy or other neurosurgical site with liquid alginate, as one example only and without limitation, sodium alginate, then adding the non-toxic reactive component: calcium chloride. Liquid sodium alginate remains non-reactive until it comes into contact with concentrated calcium chloride.

We have also tested alginate biocompatibility. Short- and long-term tissue reactivity was tested by injecting calcium alginate into the fat capsule surrounding the kidney of 32 rats weighing 300±50 g each. The rats were anesthetized with a ketamine cocktail (50 mg ketamine, 5 mg Xylazine, 1 mg PromAce) dose of 0.5 to 1 ml per animal. A 3 cm incision was made on the left side of the abdomen. The fat capsule around the left kidney was isolated. A pocket was made in the capsule, next to the kidney, and approximately 0.5 ml of alginate and 0.68 M CaCl₂.2H₂O, at a 1:1 volume ratio, was injected and polymerized. Each of the four indicated polymer types (Table 1) was injected into the kidney of two separate rats to determine the significance of the tissue reaction during a set time period (total of 8 rats per time period). The second kidney of each rat was untouched and served as a control. Separate groups of 8 rats were sacrificed after 1 day, 1 week, 3 weeks, and 9 weeks, a total of 32 rats for the entire study. Both kidneys were harvested from each rat. Tissue reactivity was first classified by visual inspection. Polymer encapsulation, organ and tissue adhesion, and tissue necrosis are strong indicators of polymer incompatibility. Visual severity classification was adopted and modified from a nonspecific, acute ASTM standard test of polymer-tissue interaction and irritation, which consists of ranking the reactivity of the kidney and surrounding tissue on a scale of 0 to 4; 0 to 1 being little or no reaction, adhesion, or encapsulation and 4 being major adhesion, encapsulation, and/or tissue necrosis.

Crude alginate exhibits significantly higher reactivity than purified alginates, and high M acid gels induce a faster immune response than high G acid gels (Table I). Overall reactivity of crude alginate is consistently high (severity of 3 to 4) independent of acid content. Purified alginate exhibits a significantly lower immune response. The overall reactivity remains consistent between the two alginic acid concentrations (severity of 1 to 2), and the high M content alginate again exhibits a faster immune response.

TABLE I
Visual severity averages and standard deviations of polymer reactivity

| | Polymer type | | | | | | | |
|---|---|---|---|---|---|---|---|---|
| Implant | | std. | | std. | | std. | | std. |
| Time (days) | CHM | dev. | CHG | dev. | PHM | dev. | PHG | dev. |
| 1 | 3.0 | 1.41 | 1.5 | 0.71 | 1.0 | 0.00 | 1.0 | 0.00 |
| 7 | 3.5 | 0.71 | 2.0 | 0.00 | 2.0 | 0.00 | 1.0 | 0.00 |
| 21 | 4.0 | 0.00 | 3.5 | 0.71 | 2.0 | 0.00 | 2.0 | 0.00 |
| 63 | 3.0 | 0.00 | 3.0 | 1.41 | 1.5 | 0.71 | 1.5 | 0.71 |

CHM = Crude, high mannuronic calcium alginate
CHG = Crude, high guluronic calcium alginate
PHM = Purified, high mannuronic calcium alginate
PHG = Purified, high guluronic calcium alginate.

The studies were expanded to determine the effect of alginate structure and purity on the resulting mechanical strength and biocompatibility. It was found that alginates with a high guluronic acid content (G/M ration>60/40) had optimal strength, polymer yield, and biocompatibility. Alginates with a molecular weight range from 50,000 g/mol to 200,000 g/mol were optimal or use as dural sealants.

In some embodiments, the invention comprises a method of sealing dura or pia mater in conjunction with surgical procedures involving the mammalian nervous system, including without limitation, the brain and spinal cord. As one example only, a craniotomy procedure involves creating an opening in the skull bone to expose the underlying contents. The clinician or researcher performs an incision in the scalp at the surgical site, and the scalp is moved to visualize the underlying bone. One or more openings are made in the skull bone with specialized instruments. The plate of bone is removed, exposing the dura mater overlying the brain tissue. Depending on the nature of the procedure, the dura may be cut, and the surface of the brain, including the pia, is exposed. The selected procedure proceeds until completed. For example, the clinician may place a microelectrode or other neuroprosthetic device in place, then proceed to close the surgical site. In accordance with the invention, a calcium alginate sealant is applied to provide a covering, inhibit and prevent migration of fluid or tissue into or out of the site, prevent invasion by infectious agents, and other purposes consistent with the invention. Aspects of the dura may or may not be closed by suture or by other means, and bone plate may be replaced.

We have discovered that calcium alginate (as one example only and without limitation, ALGEL®, used in conjunction with implanted Michigan probes (CNCT, University of Michigan), provides a suitable interface between the brain cortex and craniotomy. In some embodiments, calcium alginate acts as a transparent “window” into the brain, providing opportunities for long-term cortex visualization. In this manner, the lifetime and success of chronically implanted probes may be enhanced and extended.

In some embodiments, among others, the present invention comprises a method for applying a calcium alginate sealant to a mammalian neurosurgical site with improved ease of application. The existing art, for example, using Gelfoam, involves a considerable amount of time for application. Each piece of the material must be individually broken off and placed precisely to fill in any gaps. The time that the surface of the brain is exposed is critical and should be minimized to avoid swelling and dehydration.

By comparison, the present invention comprises calcium alginate compositions of two parts that, when mixed, creates a gel-like polymer, Thus, the application time is shortened considerably. The polymer has a low viscosity in its unreacted liquid form and therefore can be put separately into two or more different micro-syringes. As described herein, a small amount from each syringe, as one example only, a few drops, is sufficient to produce an appropriate amount of the polymer in accordance with the invention. Diffusion allows for complete mixing, and polymerization occurs almost instantaneously. Moreover, when desired, precise measurements of the mixing volumes are not critical. Unpolymerized fluid may be soaked up by appropriate means, as examples, a small cotton ball or piece of gauze. After excess fluid has been soaked up, multiple applications may be performed until the desired thickness of the polymer is established. Thus, in a few short steps and a few minutes time, the calcium alginate sealant of the present invention can be completely applied.

The present invention also comprises the use of biocompatible materials. Since reaction of the body to the surgical procedure is a primary concern, the material placed in direct contact to the brain should be carefully considered. The short and long-term tissue reactivity and biocompatibility of calcium alginate has previously been characterized and has shown to be safe in a biological system. Calcium alginate's biocompatibility characteristics make it a suitable candidate for direct contact with the brain and as a barrier to the outside environment. Once the liquid calcium alginate polymerizes, a direct pathway for infectious pathogens or toxins is eliminated. This includes the diffusion of contaminants, toxins. Solvents from dental acrylic may be applied later, as desired. As a result, highly toxic dental acrylic can be placed in direct contact with the calcium alginate with no adverse effects.

In addition, the polymer of the invention is a minimally-porous gel, and is unlikely to host tissue in-growth. In contrast, existing materials, such as Gelfoam, a porous matrix-like material, may enhance the tissue response and produce large amounts of scar-tissue growth around the implant site which can be hazardous to the implanted electrode. By comparison, using the calcium alginate of the present invention, the dural growth anchors around the polymer but does not grow toward the electrode.

We have found that the calcium alginate composition of the present invention has exemplary mechanical properties for closing up neural implant craniotomies. The mechanical strength increases with increased calcium alginate concentration. In some embodiments, without limitation, the concentration of calcium alginate used may be about 1.00 wt % to about 2.5 wt % in water (purified high-guluronic acid content alginate, apparent viscosity of about 20-200 mPas). Once the liquid calcium alginate polymerizes, the mechanical properties are sufficient in strength to apply a constant pressure to avoid brain swelling, yet still elastic enough to allow the electrode to remain ‘flexible’.

We have discovered that, in some embodiments, the invention comprises other mechanical characteristics that are suitable for neurosurgical procedures involving the implantation of electrodes. As one example only, the calcium alginate of the invention polymerizes to the shape of the rugged bone around the perimeter of the craniotomy and precisely around the shape of the electrode. The resulting gel forms a mechanical bonding interface that anchors the calcium alginate to the surrounding bone and to the electrode. The interface prevents any additional movements of the electrode relative to the brain. In this fashion, the mechanical stability of the calcium alginate also prevents the electrode from pulling out of the tissue. Previous studies have shown that if an electrode is too flexible, it tends to work its way out of the brain. This is most likely due to significant and repetitive micro-motion of the brain.

The present invention also comprises alginate-based compositions and methods that inhibit brain surface pulsation. Due to pulmonary and circulatory effects, blood flowing in the vasculature of the brain causes the cortical surface to pulsate in a rhythmic pattern. This motion is very small and usually unnoticeable when the dura is still intact. However, when the dura has been removed and the brain has room to expand and contract, this motion is significant. When formed according to the present invention, calcium alginate minimizes large brain surface pulsations that become evident once the dura is removed. Calcium alginate applies a constant pressure to the exposed surface of the brain, reducing rhythmic motion and simulating an intact dura.

The present invention also comprises alginate-based compositions and methods that, in some embodiments, may optimize histological dissection of implanted electrode. In particular, and without limitation, calcium alginate's mechanical properties are suitable for conducting histological evaluations. As one example, after the animal has been sacrificed and the brain is removed from the skull, the electrode must be cut along the ribbon cable (in the case of a thin-film flexible electrode) in order to keep the implanted portion of the electrode intact with the brain. Electrodes sealed with dental acrylic form a solid barrier that cannot be easily cut through. However, when the calcium alginate of the present invention is used between the brain and the acrylic, an accessible cavity is created. The cavity can be entered, for example, with a fine pair of dural scissors to cut the electrode. The polymer is soft enough that the dural scissors can easily cut through the polymer. This procedure is performed very delicately in order to avoid any minute movements of the electrode.

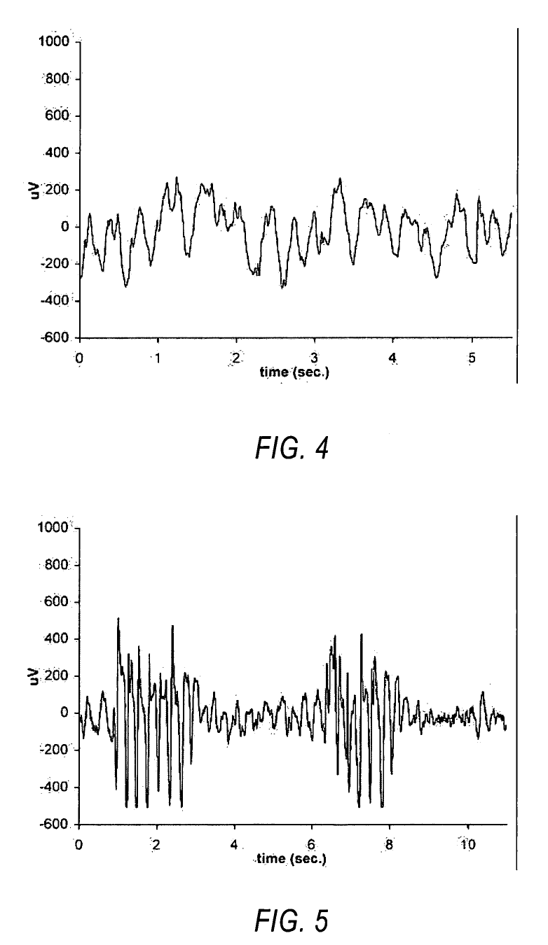

In some embodiments, the present invention comprises an alginated-based neural sealant of transparent clarity. In many neurosurgical procedures, visualization of the electrode implant site is an important aspect of the procedure. Typically, multiple still images are taken after the electrode is implanted, as shown in FIG. 6. The images show specifically where the electrode was implanted, relative to landmark vessels on the cortex. These visualizations also show how well the implantation was performed. If the brain was damaged during the preparation or if any bleeding occurred, images will capture this information for future reference. In accordance with the invention, with calcium alginate, the insertion site may continue to be visualized. Histological studies have shown that calcium alginate remains completely transparent and colorless for periods exceeding three months. Therefore, either video or still images may be taken even after calcium alginate has polymerized. In contrast, with many existing materials, for example, Gelfoam, little or no visual information is available once the craniotomy is filled. In some surgical procedures, hemorrhaging may occur after closing up yet not be realized until histology was performed. Extensive hemorrhaging on the surface of the brain can cause damage to the nearby neural tissue. The use of calcium alginate allows the surgeon to verify that no bleeding exists after the brain is completely ‘sealed’ up.

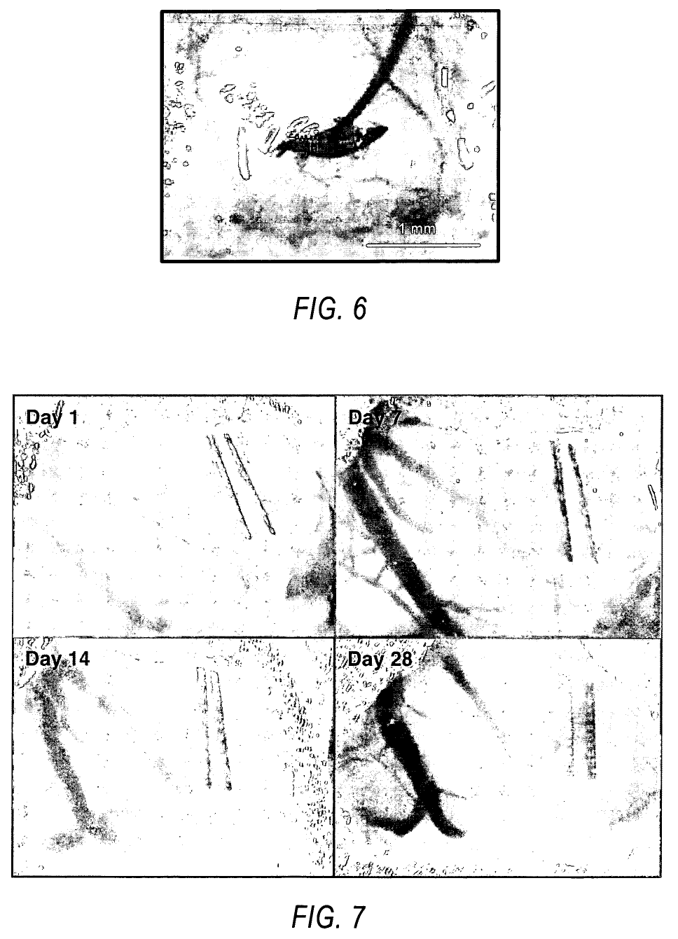

Moreover, with calcium alginate remaining completely transparent for extended periods of time, a new set of experiments can optionally be conducted in an appropriate host. With the application of calcium alginate in conjunction with a clear glass plate, a “window” into the brain can be implemented. This “window” allows for novel types of data collection from the cortex in an in vivo preparation; for example, the imaging of specific neural activity using delivery of voltaic dies, blood flow in the local vasculature using ultrasonic or laser doppler flowmetry, and local tissue response around an implanted microelectrode using 3-D confocal microscopy. Studies included the creation of a calcium alginate-coverslip interface to peer into the implant site. The window remained completely clear and imageable for over 9 weeks (FIG. 7 is an example up to 28 days). The limiting factor was the coverslip, which became scratched, making it difficult to visualize a high level of detail. However, during visualization, the tissue response of the brain around the implant could be monitored (20× magnification) and individual red blood cells flowing through the surface vessels could be visualized (50× magnification).

Finally, the calcium alginate of the present invention can be infused with anti-inflammatory drugs that can be used to further increase biocompatibility. By way of one example only, results of systemic injections of dexamethasone demonstrate a decreased immune response to implanted devices. Some of the difficulties associated with systemic drug delivery (high dosages, non-specific tissue targeting) can be alleviated by delivery of drugs directly from the implant craniotomy. Thus, in some embodiments, the calcium alginate polymer of the present invention comprises both a dural sealant and a drug delivery implant.

### EXAMPLES

Twenty-five Sprague Dawley Rats were evaluated each weighing approximately 300 grams at the time of surgery. Craniotomy locations spanned three different areas of the cortex: auditory, barrel, and motor cortex. Anesthesia was administered using intra-peritoneal injections of an anesthetic cocktail (comprised of Ketamine, Xylazine, and Acepromazine, each with concentrations of 100 mg/ml and a respective mixing ratio of 5:0.5:1). The initial dosage used was 1.5 ml/100 g body weight. This was followed by regular supplements of pure Ketamine (¼ the initial volume injected) every 60 minutes or as needed to maintain the animal in an areflexive state. Craniotomies were instituted according to techniques know in the art. The craniotomies created were rectangular in shape spanning approximately 3 mm in the anterior-posterior direction, and 2 mm in the medial-lateral direction. The electrodes were hand-inserted, and calcium alginate was applied.

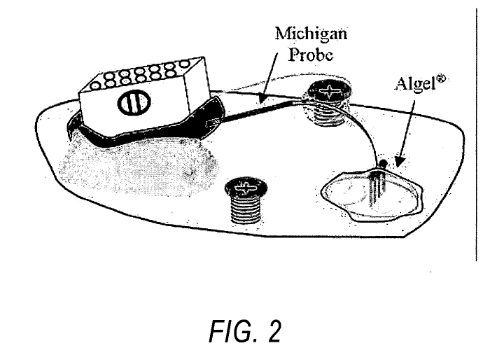

FIG. 2 illustrates a typical surgical implantation setup and this shows the superior surface of the rat skull, with three bone screws; one covered with PMMA used for fixing the Omnetics® connector (Omnetics Minneapolis, Minn., USA) to the bone, and one used for attaching a ground wire. The illustrated craniotomy is implanted with a Michigan electrode and filled with an appropriate amount of calcium alginate (as one example only and without limitation, ALGEL® (Neural Intervention Technologies, Ann Arbor, Mich.).

Since the calcium alginate is a two-component polymer, the components can be administered or applied, by way of one example only, using two separate 1 cc tuberculin syringes. Other means of application of calcium alginate or calcium chloride solution will be known to those of ordinary skill in the art. In this example, one syringe was filled with approximately 0.2 cc of liquid calcium alginate and the other with the same amount of calcium chloride. When administering the polymer, one to two drops of the liquid calcium alginate were delivered, following immediately with the same number of drops of calcium chloride. Polymerization of the calcium alginate occurs by simple diffusion of the calcium ions into the liquid calcium alginate occurring almost instantaneously. Any solution that does not polymerize was immediately absorbed up by a small piece of gauze. After this is done, the process can be repeated until several (2-5) thin layers of the polymerized calcium alginate exist that completely fill the craniotomy. FIG. 6 is a photograph taken through a surgical microscope immediately after the administering of a thin layer of calcium alginate to an implanted chronic Michigan electrode. Once the desired thickness is achieved, polymethyl methacrylate (Co-Oral-Ite Dental Mfg. Co.) is applied, to further anchor the device to the skull.

The use of calcium alginate has been shown to be mechanically stable and biocompatible for extended periods of time. We have described the application of calcium alginate in conjunction with microelectrode implants in rats (420 days), however calcium alginate has also been used for this same application in guinea pigs (90 days) and 1 cat (100 days). In addition calcium alginate has been evaluated for other applications in several other species including rabbits, sheep and swine. The calcium alginate did not show any type of adverse reaction or tissue response in any of the listed cases.

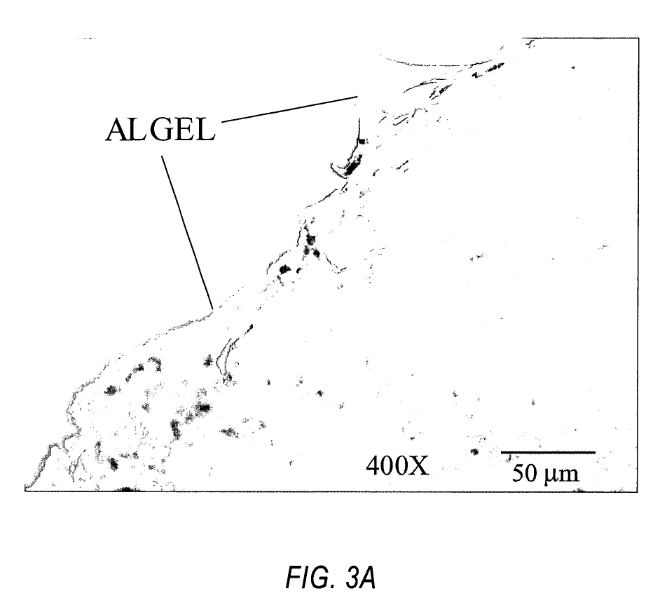

FIGS. 3A-C show histology images of rat cortex protected by calcium alginate sealant in accordance with the present invention, with the minimal tissue reaction at or near the probe implant sites (from 3 month, 6 month, and 9 month implanted rats). FIG. 3A shows normal pia surface with traces of the remaining alginate layer (3 month implant). FIG. 3B shows an implant probe track and pial layer visible with no adverse tissue response (6 month implant). The cortical surface is intact and pia shows no thickening or reaction. FIG. 3C shows pia mater and cortex still intact after 9 month implantation.

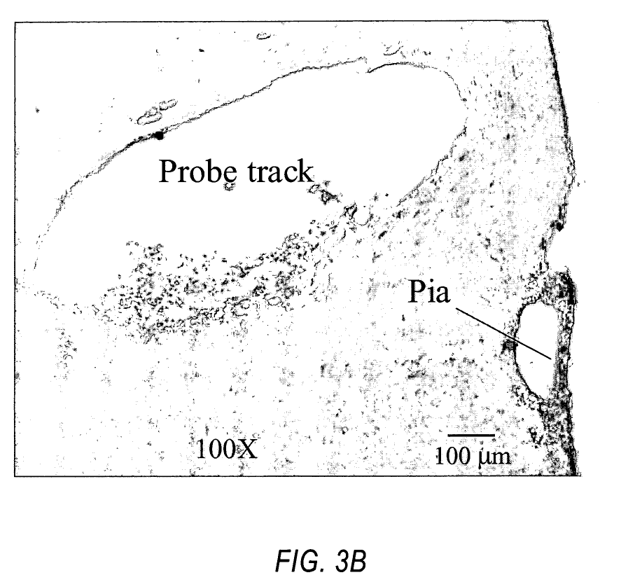

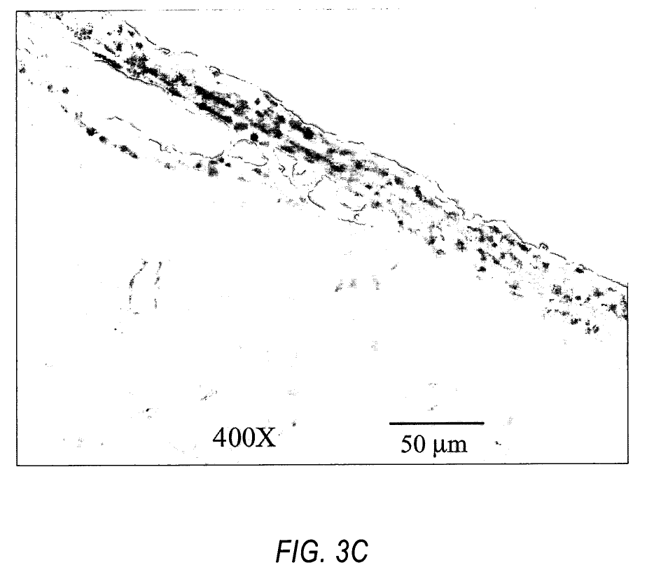

EEG data show that calcium alginate and calcium chloride of the present invention do not elicit abnormal neural activity when placed on the surface of the brain. Fifteen rats treated with alginate dural sealant and monitored exhibited normal EEG waves (FIG. 4) that underwent predictable shifts corresponding to the controlled levels of anesthesia. No abnormal neural activity was seen after application of alginate and calcium chloride directly to the cortical surface. One rat acted as a seizure control and was treated with tranexamic acid (tAMCA), a chemical known to induce seizures in cats and rats when applied topically to the cortex or in combination with fibrin sealants. Five minutes post-application of tAMCA, convulsive behavior and muscle jerks manifested first in the left HL, then in the left side of the torso, and then spread to include the front leg (FL), the head, and the entire left side. (FIG. 5). During the seizures, the animal underwent a rigid posture while the head and FL showed irregular spasms and tremors. The seizures continued with strong jerks of the left torso, HL, and head. The corresponding EEG showed significant spiking during the seizing periods (FIG. 5). In contrast, no spiking or abnormal EEG signals were seen in conjunction with use of the present invention. No abnormal EEG spikes, twitching, or convulsive behavior was seen at any time in the fifteen rats treated with alginate during the 2 hour EEG recordings. Thus, the present invention comprises an effective artificial dura material with enhanced material properties and biocompatibility characteristics.

While the present invention has been particularly shown and described with reference to the foregoing preferred and alternative embodiments, it is to be understood that this is by way of illustration and not of limitation, and various alternatives to the embodiments of the invention described herein may be employed in practicing the invention without departing from the spirit and scope of the invention as defined in the following claims, which should be construed as broadly as the prior art will permit.

1. A method of sealing the site of a neurosurgical procedure comprising:
applying a purified alginate solution to the site of a neurosurgical procedure in a mammal, and
adding a calcium chloride solution to form a polymer.

2. The method of claim 1, wherein the neurosurgical procedure comprises implantation of a neural probe.

3. The method of claim 1, wherein the site of the neurosurgical procedure is the central nervous system.

4. The method of claim 1, wherein the site of the neurosurgical procedure is the brain.

5. The method of claim 1, wherein the site of the neurosurgical procedure is the spinal cord.

6. The method of claim 1, wherein the mammal is a human being.

7. The method of claim 1, wherein the purified alginate solution is comprised of alginates with a molecular weight range from about 50,000 g/mol to about 200,000 g/mol.

8. The method according to claim 1, wherein the concentration of purified alginate in the purified alginate solution is about 1.00 wt % to about 2.5 wt % in water.

9. The method of claim 1, wherein the purified alginate is purified high-guluronic acid content alginate with apparent viscosity of about 20 mPas to about 200 mPas.

10. The method of claim 1, wherein the purified alginate solution further comprises one or more therapeutic drugs.

11. The method according to claim 1, wherein the concentration of calcium chloride in the calcium chloride solution is about 1.00 wt % to about 30.0 wt %.

12. A method of visualizing the site of a neurosurgical procedure comprising:
applying a purified alginate solution to the site of a neurosurgical procedure in a mammal, and
adding a calcium chloride solution to form a polymer.

13. The method of claim 12, wherein the neurosurgical procedure comprises implantation of a neural probe.

14. The method of claim 12, wherein the site of the neurosurgical procedure is the central nervous system.

15. A method of stabilizing a neural probe implanted at the site of a neurosurgical procedure comprising:
applying a purified alginate solution to the site of a neurosurgical procedure in a mammal, where the neurosurgical procedure comprises implantation of a neural probe, and
adding a calcium chloride solution to form a polymer.

16. The method of claim 15, wherein the site of the neurosurgical procedure is the central nervous system.
